(19) United States
(12) Patent Application Publication
(10) Pub. No.: US 2009/0198087 A1
(43) Pub. Date: Aug. 6, 2009

(54) Method and Device for Providing a Reducing Agent Precursor

(75) Inventor: Rolf Bruck, Bergisch Gladbach (DE)

Correspondence Address: LERNER GREENBERG STEMER LLP, P O BOX 2480, HOLLYWOOD, FL, 33022-2480, US

(73) Assignee: EMITEC GESELLSCHAFT FUR EMISSIONSTECHNOLOGIE MBH, Lohmar (DE)

(21) Appl. No.: 12/417,193

(22) Filed: Apr. 2, 2009

Related U.S. Application Data
(63) Continuation of application No. PCT/EP2007/059802, filed on Sep. 17, 2007.

(30) Foreign Application Priority Data
Oct. 2, 2006 (DE) 10 2006 047 018.4

(51) Int. Cl. C07C 273/02; A61L 2/04

(52) U.S. Cl. 564/63; 422/307

(57) ABSTRACT

A method for providing a reducing agent precursor includes heating a quasi-closed evaporator volume, delimited by a wall and at least partially filled with a solution of a reducing agent precursor, to a reserve temperature of the solution to produce an atmosphere of a vapor including at least one reducing agent precursor at a reserve pressure above ambient pressure. The vapor is extracted on demand by utilizing a pressure difference between the reserve pressure and an ambient pressure. The method and a device provide reducing agent precursors that can especially be used in the selective catalytic reduction of nitrogen oxides. The method and the device allow easy dosing since, due to the pressure gradient between the vapor and an extraction line, no other conveying device is required when the vapor, which contains at least one reducing agent precursor, is dosed to a hydrolysis catalytic converter.

### CROSS-REFERENCE TO RELATED APPLICATION

This is a continuing application, under 35 U.S.C. § 120, of copending International Application No. PCT/EP2007/059802, filed Sep. 17, 2007, which designated the United States; this application also claims the priority, under 35U.S.C. § 119, of German Patent Application No. 10 2006 047 018.4, filed Oct. 2, 2006; the prior applications are herewith incorporated by reference in their entirety.

### BACKGROUND OF THE INVENTION

### Field of the Invention

The present invention relates to a method and a device for providing a reducing agent precursor, in particular urea. The preferred field of use of the present invention is the delivery of a reducing agent precursor for generating a reducing agent for selective catalytic reduction (SCR) in the automotive field.

The exhaust gases of internal combustion engines contain substances, the emission of which into the environment is not desirable. In many countries, there are limit values to which the emissions of internal combustion engines must adhere with regard to certain substances in the exhaust gas. Those substances also include nitrogen oxides (NOₓ), the emission of which is limited in many countries by legally stipulated limit values. In order to adhere to the limit values and to generally be able to lower the nitrogen oxide emissions, it is possible firstly to use engine-internal measures and secondly to use exhaust-gas aftertreatment measures. In terms of exhaust-gas aftertreatment measures, the selective catalytic reduction of the nitrogen oxides with a reducing agent, such as for example ammonia (NH₃), which acts selectively on nitrogen oxides, has proven to be particularly effective. It is often not the reducing agent itself which is stored but rather a reducing agent precursor, such as for example urea for the reducing agent ammonia. A reducing agent precursor is to be understood to mean a substance which can cleave the reducing agent or which can react to form the reducing agent. That often occurs through the use of thermolysis and/or hydrolysis on a correspondingly formed hydrolysis catalytic converter. There are often problems in the provision of the reducing agent precursor for thermolysis and/or hydrolysis.

### SUMMARY OF THE INVENTION

It is accordingly an object of the invention to provide a simple method and a simple device for providing a reducing agent precursor, which overcome the hereinafore-mentioned disadvantages of the heretofore-known methods and devices of this general type.

With the foregoing and other objects in view there is provided, in accordance with the invention, a method for providing a reducing agent precursor. The method comprises:
A) heating a quasi-closed evaporator volume, delimited by a wall and at least partially filled with a solution of at least one reducing agent precursor, to a reserve temperature above an evaporation temperature of the solution to form an atmosphere of a vapor including at least one reducing agent precursor at a reserve pressure above an ambient pressure; and
B) extracting the vapor on demand by utilizing a pressure difference between the reserve pressure and the ambient pressure.

A quasi-closed evaporator volume is to be understood to mean a volume through which flow basically does not pass, but rather in which a certain quantity of the solution is stored statically. A quasi-closed evaporator volume is also to be understood in particular to mean that only a small proportion of the surface of the wall which delimits the evaporator volume is formed by openings. A quasi-closed evaporator volume is thus to be understood in particular to mean an evaporator volume in which at least 90% of the surface is closed. A value of at least 95% of the surface of the wall which delimits the quasi-closed evaporator volume is preferable in this case. The remaining openings can in particular be closed off, in such a way that the openings are in particular open only during the extraction of the vapor or during the supply of solution, and are otherwise closed. The reserve temperature is to be understood in particular to mean the temperature of the solution. If appropriate, the temperature of the wall of the evaporator volume may lie slightly higher than the reserve temperature, such that the solution is at the reserve temperature. Within the context of the present invention, vapor is to be understood to mean a gas which includes at least one reducing agent precursor and which has been generated through the use of evaporation of the solution.

A quasi-closed evaporator volume has the effect that, during heating to the reserve temperature, thermodynamic equilibrium is generated between the vapor above the liquid and the liquid. Depending on the construction, that is to say in particular depending on the volume of the evaporator volume and depending on the temperature, a predefinable reserve pressure is set, wherein it is ensured through the use of the construction of the evaporator volume that the reserve pressure lies above the ambient pressure. The reserve pressure is in particular the pressure of the vapor when the openings of the quasi-closed evaporator volume are closed, or the equilibrium pressure under the given conditions.

Through the use of the method according to the invention, it is possible in a very simple manner for the reducing agent precursor to be dosed into a hydrolysis catalytic converter which may be provided. In this case, the pressure gradient between the evaporator volume and the environment is used for dosing and for delivery.

Extraction on demand is to be understood in particular to mean that precisely that quantity of vapor which contains the quantity of reducing agent required for the selective catalytic reduction of the nitrogen oxides is extracted. The extraction is realized in particular by activating a correspondingly constructed valve.

In accordance with another mode of the method of the invention, the solution of at least one reducing agent precursor is supplied to the evaporator volume on demand. This may mean in particular that the filling level of the evaporator volume, that is to say the position of the liquid level, is monitored and, in the event of a predeterminable limit value being undershot, the evaporator volume is refilled for example by pumping in more solution. In particular, it is also possible for solution not to be pumped in if the presently introduced and measured heating power lies above a predefinable limit value, since energy is then required for the evaporation, which energy is not available for heating freshly introduced solution.

In accordance with a further mode of the method of the invention, the reserve temperature is 140° C. to 180° C.

It is particularly preferable in this case for the method to be implemented in such a way that the reserve temperature is set below 153° C., preferably below 150° C. Those temperatures have proven to be particularly advantageous since practically no secondary reactions of urea into other products have been observed. The reserve pressure in the static case, that is to say when the openings are closed, is preferably in a range of from 3 bar to 10 bar, preferably 4 to 6 bar, and is particularly preferably approximately 5 bar.

In accordance with an added mode of the method of the invention, the vapor is heated after step B).

In this way, it is possible in particular to provide that, as flow passes through a hydrolysis catalytic converter downstream of the evaporator volume, no cooling of the hydrolysis catalytic converter takes place. It is even possible for heating of the hydrolysis catalytic converter not to take place if the temperature to which the vapor is heated after step B) is correspondingly selected or regulated in such a way that it is always ensured that the temperature of the hydrolysis catalytic converter does not fall below the light-off temperature. In this case, it is particularly advantageous for the vapor to be heated to temperatures from 250° C. to 550° C., particularly preferably temperatures of 350 to 450° C.

With the objects of the invention in view, there is also provided a device for providing a reducing agent precursor. The device comprises a wall delimiting a quasi-closed evaporator volume configured to be filled with a solution of at least one reducing agent precursor. At least one heating device is provided for at least partially heating the wall and for evaporating the solution disposed in the evaporator volume. The evaporator volume has an extraction opening for extraction of a vapor including at least one reducing agent precursor.

The device according to the invention particularly advantageously makes it possible for the reducing agent precursor to be dosed, by closing and opening the extraction opening, for example to a hydrolysis catalytic converter, which is provided downstream, in a simple manner utilizing a pressure gradient. The device according to the invention may in particular be used for carrying out the method according to the invention.

In accordance with another feature of the invention, in this connection, it has proven to be particularly advantageous if the extraction opening or an extraction line which is situated downstream of the extraction opening can be reversibly closed off, for example through the use of a correspondingly constructed valve. The valve may be controlled and actuated through the use of a control unit which can preferably also activate the heating device.

In accordance with a further feature of the device of the invention, a supply opening for the supply of the solution is provided.

The supply opening is in particular constructed in such a way that, as the solution is dosed in, the solution cannot leave directly through the extraction opening, if the latter is open, without evaporation of the evaporator volume.

In accordance with an added feature of the device of the invention, the supply opening can be assigned a supply vector in the direction in which the solution is supplied, while the extraction opening can be assigned an extraction vector in the direction in which the gas can be extracted, with the extraction and supply vectors enclosing an acute angle, a right angle or an obtuse angle with respect to one another.

In accordance with a concomitant feature of the device of the invention, the heating device includes at least one self-regulating electrical resistance heater having a regulating temperature which lies in a range of from 140° C. to 180° C.

A self-regulating resistance heater is to be understood in particular to mean a PTC (positive temperature coefficient) resistor which may be produced in particular from a titanate ceramic. A self-regulating resistance heater of that type has a regulating temperature. In the event of a deviation from that temperature, a change takes place in the resistance heater. It is thus possible to realize self-regulating heating. In order to avoid blockages in the system, the regulating temperature is preferably selected in such a way that reserve temperatures of less than 153° C., preferably of less than 150° C., and in particular in a range between 145° C. and 150° C., are preferable.

Within the context of the present application, a reducing agent precursor is to be understood in particular to mean at least one of the following substances and derivatives of these substances:
a) urea ((NH₂)₂CO);
b) ammonium formate (HCOONH₄);
c) ammonium carbamate (H₂NCOONH₄);
d) ammonium carbonate ((NH₄)₂CO₃);
e) ammonium bicarbonate (NH₄HCO₃);
f) ammonium oxalate ((NH₄)₂(C₂O₄));
g) ammonium hydroxide (NH₄OH);
h) cyanic acid (HOCN);
i) cyanuric acid (C₃H₃N₃O₃); and
j) isocyanic acid (HNCO).

The details disclosed within the context of this invention for the method can be transferred and applied in the same way to the device according to the invention. The details disclosed within the context of this invention for the device according to the invention can be transferred and applied in the same way to the method according to the invention.

Other features which are considered as characteristic for the invention are set forth in the appended claims.

Although the invention is illustrated and described herein as embodied in a method and a device for providing a reducing agent precursor, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings, without the invention being restricted to the exemplary embodiments and details shown in the figures.

### BRIEF DESCRIPTION OF THE SEVERAL VIEWS OF THE DRAWING

FIG. 1 is a fragmentary, diagrammatic, partly sectional view of a first exemplary embodiment of a device according to the invention;

FIG. 2 is a view similar to FIG. 1 of a second exemplary embodiment of a device according to the invention; and

FIG. 3 is a view similar to FIGS. 1 and 2 of a third exemplary embodiment of a device according to the invention.

### DETAILED DESCRIPTION OF THE INVENTION

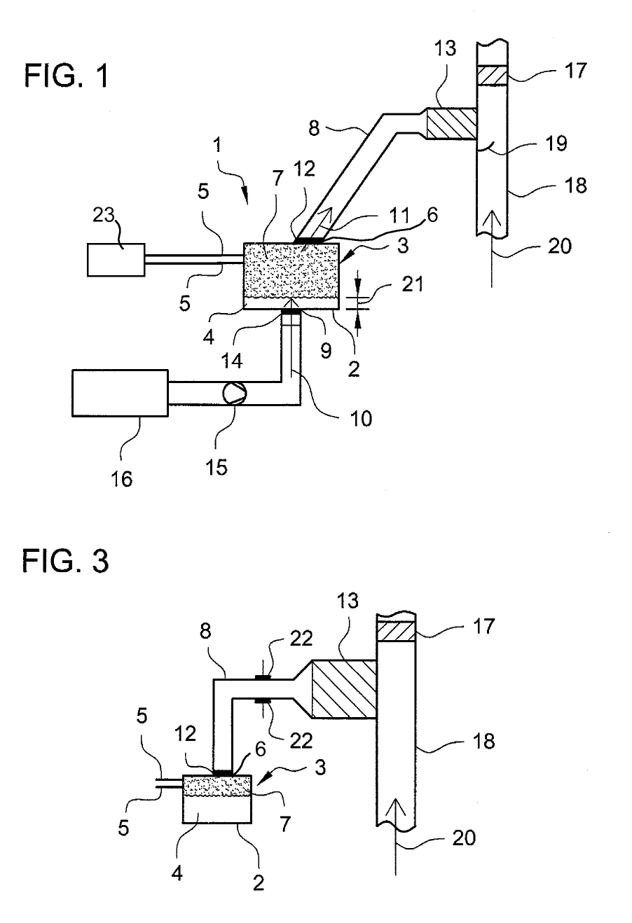

Referring now to the figures of the drawings in detail and first, particularly, to FIG. 1 thereof, there is seen a diagrammatic illustration of a device 1 for providing a reducing agent precursor. The device 1 includes a quasi-closed evaporator volume 3 which is delimited by a wall 2. The evaporator volume 3 is at least partially filled with a solution 4 of at least one reducing agent precursor, in particular of urea and if appropriate of ammonium formate. The wall 2 can be at least partially heated by at least one heating device 5, which is shown herein as connecting wires of at least one self-regulating electrical resistance heater 23. A closable extraction opening 6 is formed in the wall 2. A vapor 7, which is generated above the solution 4 and which is indicated by corresponding dots in the figure, can be extracted through the extraction opening 6. In this case, during operation, the wall 2 and/or the evaporator volume 3 is/are heated to a reserve temperature, such that the vapor 7 is at a reserve pressure which lies above the ambient pressure, for example a pressure in an extraction line 8 which is connected to the extraction opening 6.

In the present first exemplary embodiment, a supply opening 9 is also provided. The supply opening 9 can be closed off in a reversible and activatable manner. The supply opening 9 is assigned a supply vector 10 which denotes the direction in which the solution 4 is supplied. Furthermore, the extraction opening 6 is assigned an extraction vector 11 which specifies the direction in which the vapor can be extracted. The extraction and supply vectors enclose an acute or obtuse angle in the present exemplary embodiment. In other words, the vector product of the extraction vector 11 and the supply vector 10 is non-zero. This ensures that the solution 4 which is introduced cannot leave the evaporator volume 3 directly through the extraction opening 6 without evaporating. In the present example, the extraction opening 6 can be reversibly closed off through the use of an extraction valve 12. It is possible, through the use of an activation of the extraction valve 12, for the reducing agent precursor or the vapor 7 including at least one reducing agent precursor to be dosed to a hydrolysis catalytic converter 13 provided downstream. The filling process can be controlled or regulated through the use of a supply valve 14 which reversibly closes off the supply opening 9. As it is supplied, the solution 4 is delivered through the use of a pump 15 which delivers the solution 4 from a reservoir 16 through the supply opening 9 and into the evaporator volume 3.

An at least partial hydrolysis of the reducing agent precursor to form a reducing agent takes place in the hydrolysis catalytic converter 13, wherein a gas flow which includes a corresponding reducing agent can be introduced into an exhaust line 18 upstream of an SCR catalytic converter 17. The supply or the connection of the hydrolysis catalytic converter 13 to the exhaust line 18 lies in a flow shadow of a guide plate 19. It is possible to effectively prevent exhaust gas from infiltrating into the hydrolysis catalytic converter 13 through the use of the guide plate 19. In this way, it is possible for an exhaust-gas flow 20, which flows through the exhaust line 18, to undergo a reduction of its nitrogen oxide proportion in the SCR catalytic converter 17 through the use of selective catalytic reduction of the nitrogen oxides, with the reducing agent leaving the hydrolysis catalytic converter 13. An at least partial thermolysis of the reducing agent precursor may take place in the evaporator volume 3 itself or in a further heating stage situated downstream of the evaporator volume 3.

The supply of the solution 4 into the evaporator volume 3 is carried out by monitoring a solution level 21. If the solution level 21 falls below a predefinable minimum value, the solution 4 is supplied into the evaporator volume 3.

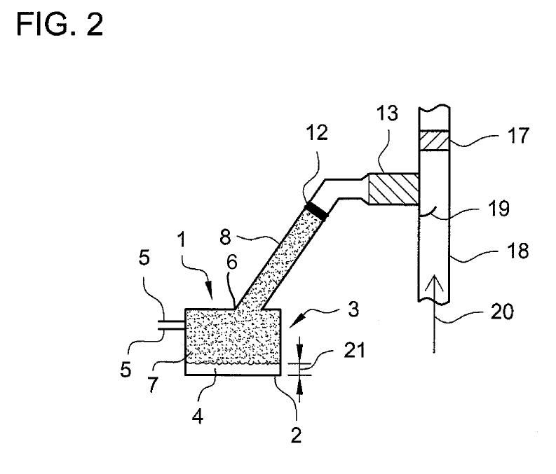

FIG. 2 shows a second exemplary embodiment of the device according to the invention, with identical parts having been provided with the same reference symbols as in FIG. 1, and with reference being made to the description regarding FIG. 1. In contrast to the first exemplary embodiment, no reservoir 16 is provided in this case. In fact, the entire reservoir is heated, in such a way that the evaporator volume 3 constitutes the entire reservoir of reducing agent precursor solution. The evaporator volumes 3 may, for example, be constructed to be exchangeable, in such a way that instead of a filling process, it is merely necessary to exchange a cartridge. In this case, it is also the case that the extraction valve 12 is formed not directly at the outlet of the extraction opening 6 but rather downstream in the extraction line 8. In this way, the evaporator volume 3 is enlarged by that volume of the extraction line 8 which is situated within or on the same side of the extraction valve 12. The embodiment according to FIG. 2 has the advantage that it is possible to dispense with a further reservoir 16 and the pump 15.

FIG. 3 diagrammatically shows a third exemplary embodiment of a device 1 according to the invention. In contrast to the second exemplary embodiment, second heating devices 22 are provided in this case. The vapor 7 can be heated further through the use of the second heating devices 22. It is preferable in this case for the vapor 7 to be heated to a temperature of 250 to 550° C. This has the result that the hydrolysis catalytic converter 13 cannot be cooled by the vapor 7 and, if appropriate, an at least partial thermolysis of the reducing agent precursor to form reducing agent takes place.

The present invention provides a method and a device 1 for providing reducing agent precursors which can be used in particular for the selective catalytic reduction of nitrogen oxides. The method according to the invention and the device 1 according to the invention make it possible for dosing to be carried out in a simple manner since, for dosing the vapor 7 which contains at least one reducing agent precursor into the hydrolysis catalytic converter 13, it is possible to dispense with a further delivery device because there is a pressure gradient between the vapor 7 and the extraction line 8.

1. A method for providing a reducing agent precursor, the method comprising the following steps:
A) heating a quasi-closed evaporator volume, delimited by a wall and at least partially filled with a solution of at least one reducing agent precursor, to a reserve temperature above an evaporation temperature of the solution to form an atmosphere of a vapor including at least one reducing agent precursor at a reserve pressure above an ambient pressure; and
B) extracting the vapor on demand by utilizing a pressure difference between the reserve pressure and the ambient pressure.

2. The method according to claim 1, which further comprises supplying the solution of at least one reducing agent precursor to the evaporator volume on demand.

3. The method according to claim 1, wherein the reserve temperature is 140° C. to 180° C.

4. The method according to claim 1, which further comprises heating the vapor after step B).

5. The method according to claim 1, which further comprises heating the wall uniformly.

6. A device for providing a reducing agent precursor, the device comprising:
a wall delimiting a quasi-closed evaporator volume configured to be filled with a solution of at least one reducing agent precursor;
at least one heating device for at least partially heating said wall and for evaporating the solution disposed in said evaporator volume; and
said evaporator volume having an extraction opening for extraction of a vapor including at least one reducing agent precursor.

7. The device according to claim 6, wherein said extraction opening can be reversibly closed off.

8. The device according to claim 6, wherein said evaporator volume has a supply opening for supplying the solution.

9. The device according to claim 8, wherein:
said supply opening is assigned a supply vector in a direction in which the solution is supplied;
said extraction opening is assigned an extraction vector in a direction in which the vapor can be extracted; and
said extraction and supply vectors mutually enclose one of an acute angle, a right angle or an obtuse angle.

10. The device according to claim 6, wherein said at least one heating device includes at least one self-regulating electrical resistance heater having a regulating temperature lying in a range of from 140° C. to 160° C.
